(19) United States
(12) Patent Application Publication
(10) Pub. No.: US 2020/0330792 A1
(43) Pub. Date: Oct. 22, 2020

(54) BlueGuard antibacterial wand

(71) Applicants: Patrick H. McCray, Ponte Vedra Beach, FL (US); Robert B. Davis, Ponte Vedra Beach, FL (US); James E. LeBlanc, Ponte Vedra, FL (US)

(72) Inventors: Patrick H. McCray, Ponte Vedra Beach, FL (US); Robert B. Davis, Ponte Vedra Beach, FL (US); James E. LeBlanc, Ponte Vedra, FL (US)

(21) Appl. No.: 16/035,618

(22) Filed: Jul. 14, 2018

Related U.S. Application Data
(60) Provisional application No. 62/538,019, filed on Jul. 28, 2017.

(51) Int. Cl. A61N 5/06

(52) U.S. Cl. CPC A61N 5/0624; A61N 5/062; A61N 2005/0651; A61N 2005/0644; A61N 2005/0632

(57) ABSTRACT

The BlueGuard Antibacterial Wand was developed to address environmental pathogens control for Humans and Animals. The Antibacterial Wand is used in the name due to the mode of application of the anti-bacterial effect of the specific light wave of the Blue Light Spectrum that has been proven to be therapeutic in that it kills a wide array of Pathogens, Viruses, Fungai, Spores & Mold, and Bacteria, and is one of the many uses for this product in a single Photobiomodualation application.

The Wand uses Light Emitting Diodes (LEDs) of specific wavelengths (measured in nanometers) to provide both red light for tissue and collagen re-growth, and blue light for bacteriostatic benefits together in a single unit. The Wands can be made in wide array of different shapes, designs, and sizes so it can be a hand-held source of therapeutic LED light.

This product may be utilized in any wound healing scenario where both tissue regeneration/growth and bactericidal control of bacteria, viral, fungal, &/or spore pathogens are needed.

The unique features of this product are the arrangement of the diodes in a mixture that provides the therapeutic light for the benefits claimed.

Background information & support data (white paper) is incorporated in the Specification portion of the Invention.

The Wand is designed to kill various pathogenic bacteria, including MRSA, and eradicate Viruses, including Coronavirus-19, by exposing the pathogenic bacteria and viruses to visible light having a specific wavelength range (measured in nanometers).

The invention, The BlueGuard Antibacterial Wand, is a handheld unit with an array of diodes (number may vary between 6-40 diodes depending on model of unit):

The wand may be modified to include any type of lighting device or fixture.

The BlueGuard Antibacterial Wand to have an array of diodes in the nanometer ranges of 400-475 (for bacteria killing and virus eradication) & 600-670 (for tissue re-growth and wound healing) nanometers in any combination and numbers. The combination of these specific nanometer ranges provides for infection control and wound healing. Units may also have diodes (Kelvin temperature range of 2500 to 5000) added to improve illumination based upon the application desired.

### DESCRIPTIONS OF DRAWINGS

FIG. 1 (Front View):

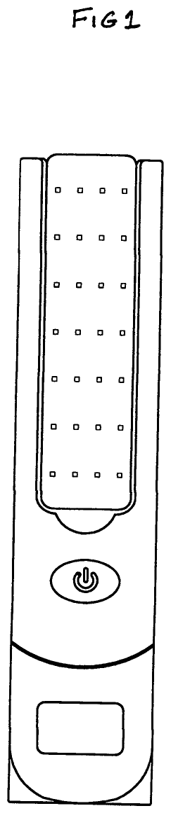

The BlueGuard Antibacterial Wand is battery operated, portable and is both rechargeable and corded.

FIG. 2 (Front View):

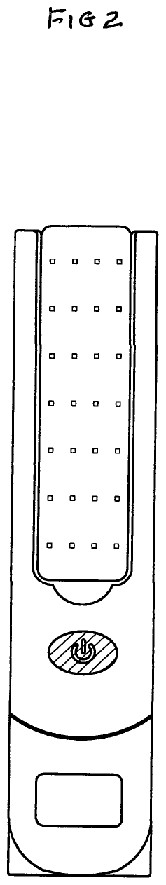

The BlueGuard Antibacterial Wand has an independent On/Off button.

FIG. 3 (Front View):

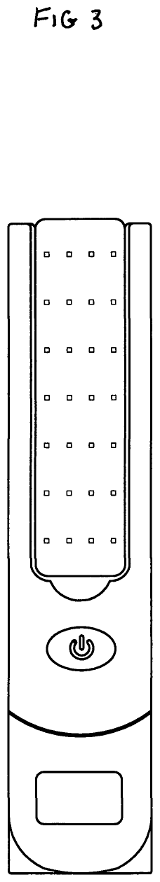

Blue Wavelength diodes, 4 rows, (400-475 nm) provides specifically to kill MRSA, and for germicidal effects on other pathogenic bacteria, viruses, swelling and infections of soft tissue wounds, trauma, and swelling.

Red Wavelength diodes, 3 Rows, (600-670 nm) provides healing effects for skin lesions of soft tissue wounds, trauma, and swelling.

FIG. 4 (Rear View Charging Port):

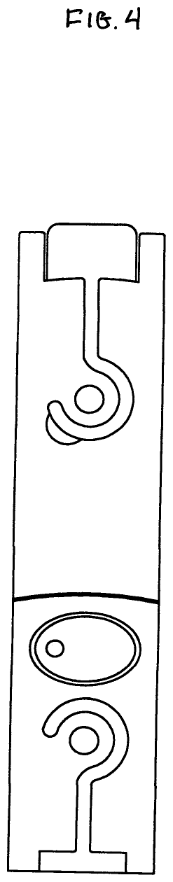

FIG. 5 (Side View):

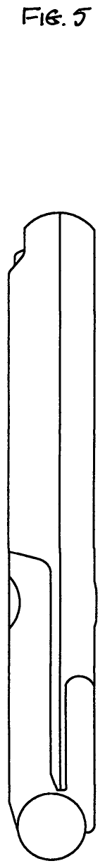

FIG. 6 (Charger):

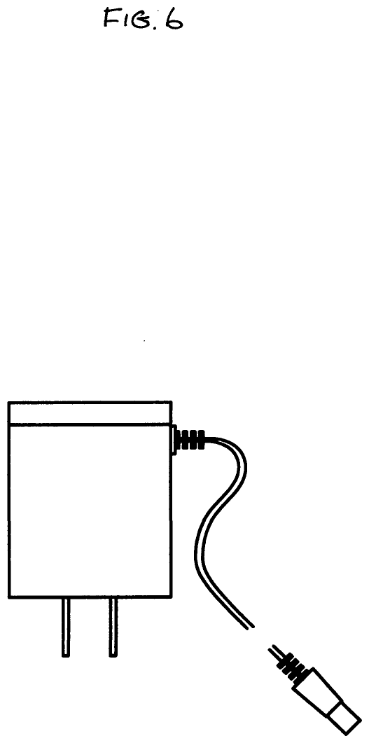

110-volt charging cable. Connects to rear charging port, and also to a 110-wall outlet.

1: Bacteriostatic,

2: Bactericidal

3: Tissue Re-generation in a hand-held Light Wand.

4: Bacteria Killing (MRSA, Staph and other Pathogenic Bacteria), Listeria, Salmonella)

5: Virus Eradication: (Flu Viruses, Varicella (VZV), Herpes Viruses, Human Immunodeficiency Virus (HIV), Shingles, Coronaviruses (COV's including CoVid-19)

6: Pain Relieving

7: Acne

8: Arthritis

9: Bursitis

10: Back Pain

11: Burns

12: Cold sores

13: Knee Pain

14: Rotator Cuff

15: Soft Tissue Injuries

16: Wound Healing: diabetic wounds, open wounds, spider bites, etc.
